(19) United States
(12) Patent Application Publication
(10) Pub. No.: US 2013/0171659 A1
(43) Pub. Date: Jul. 4, 2013

(54) METHODS OF PROGNOSIS AND DIAGNOSIS OF RHEUMATOID ARTHRITIS

(71) Applicants: Kaori Morota, Chiba-Ken (JP); Ryotaro Chiba, Chiba-Ken (JP); Rika Sekiya, Chiba-Ken (JP)

(72) Inventors: Kaori Morota, Chiba-Ken (JP); Ryotaro Chiba, Chiba-Ken (JP); Rika Sekiya, Chiba-Ken (JP)

(73) Assignee: ABBOTT JAPAN CO., LTD., Chiba-Ken (JP)

(21) Appl. No.: 13/728,003

(22) Filed: Dec. 27, 2012

Related U.S. Application Data
(60) Provisional application No. 61/585,393, filed on Jan. 11, 2012.
(60) Provisional application No. 61/580,737, filed on Dec. 28, 2011.

(51) Int. Cl. G01N 33/68

(52) U.S. Cl. CPC G01N 33/6863; G01N 33/6893
USPC 435/7.4; 435/7.92

(57) ABSTRACT

Provided are methods of diagnosing rheumatoid arthritis in a patient by detecting the presence and/or amount of a biomarker of rheumatoid arthritis in a sample from the patient. The methods and biomarkers may be used to develop an accurate prognosis for a patient having rheumatoid arthritis or suspected of having rheumatoid arthritis, or to accurately diagnose a patient having, or suspected of having rheumatoid arthritis. The methods and biomarkers may be used to identify and/or classify a patient as a candidate for a rheumatoid arthritis therapy.

### CROSS-REFERENCE TO RELATED APPLICATIONS

This application claims priority to U.S. Provisional Patent Application No. 61/585,393, filed on Jan. 11, 2012 and U.S. Provisional Patent Application No. 61/580,737, filed on Dec. 28, 2011, the contents of each of which are herein fully incorporated by reference.

### TECHNICAL FIELD

The disclosure relates to methods for determining a prognosis, diagnosis, or risk identification of rheumatoid arthritis in a patient by detecting a biomarker in the patient as well as determining amounts thereof. The biomarkers may be used to identify a patient with rheumatoid arthritis, identify a patient as a candidate for rheumatoid arthritis therapy, to classify a patient's risk of developing rheumatoid arthritis, or to classify a patient's risk of progression of rheumatoid arthritis, as well as to determine a diagnosis, prognosis, or a treatment regimen.

### BACKGROUND

Rheumatoid arthritis is a common, systemic autoimmune disease affecting 0.5-1% of the population. It is characterized by chronic inflammation of the synovium, which commonly leads to progressive joint destruction and, in most cases, to disability and reduction of quality of life. Rheumatoid arthritis is estimated to reduce lifespan from 5 to 10 years. Indicators that have been associated with higher mortality and poor prognosis include early disease onset, extended disease duration, other health problems, and characteristics of severe rheumatoid arthritis (e.g., poor functional ability, poor overall health, extensive joint damage, and involvement of organs other than the joints). Evidence gained over the last few years suggests that aggressive therapy given early in the disease has the greatest therapeutic potential. Thus, methods that provide for an earlier and/or more accurate diagnosis of rheumatoid arthritis are desirable because such methods can allow for early therapeutic intervention and can improve patient outcomes (e.g., quality of life). Several biomarkers including Rheumatoid Factor (RF), C-reactive protein (CRP), matrix metalloproteinase-3 (MMP3), mutated citrullinated vimentin (MCV), and anti-cyclic citrullinated peptide antibody (Anti-CCP) have been associated with rheumatoid arthritis and are used to diagnose the disease. However, these markers lack the desirable sensitivity and specificity that is needed for the accurate and/or early diagnosis of rheumatoid arthritis in patients.

### SUMMARY OF THE INVENTION

In one aspect, the disclosure provides a method for providing a diagnosis, prognosis or risk classification of a subject having or at risk of having rheumatoid arthritis, the method comprising determining the concentration of thymus and activation-regulated chemokine (TARC/CCL17) in a biological sample from the subject, and comparing the TARC/CCL17 concentration in the sample to a reference TARC/CCL17 concentration value, wherein when the TARC/CCL17 concentration in the sample is greater than the reference TARC/CCL17 concentration value, the concentration indicates the subject has rheumatoid arthritis or has an increased risk of having or developing rheumatoid arthritis. In embodiments, the method can further comprise detecting at least one additional biomarker of rheumatoid arthritis in the sample. In embodiments of the method, providing a diagnosis can be providing a diagnosis of rheumatoid arthritis. In other embodiments of the method, providing a prognosis can be determining rheumatoid arthritis severity, or can be determining the likelihood (or risk) that the subject will develop rheumatoid arthritis.

The method may further comprise the assessment of at least one additional biomarker of rheumatoid arthritis selected from the group consisting of RF, anti-CCP, anti-MCV, MMP3, and CRP. Assessment of the additional biomarker may comprise, for example, measuring the concentration of the biomarker in the biological sample from the subject, or may comprise a clinical evaluation of the subject. For an additional biomarker assessed by measuring the concentration of the biomarker in the biological sample from the subject, the method may further comprise comparing the measured concentration of the at least one further biomarker with a reference value for the biomarker. The reference value for the additional biomarker can be the biomarker concentration of a control sample, a biomarker cutoff value, or a median concentration of a plurality of control samples from a group of control subjects.

In one aspect, the disclosure provides a method for identifying a subject as a candidate for a rheumatoid arthritis therapeutic regimen, the method comprising determining the concentration of TARC/CCL17 in a biological sample from the subject, and comparing the TARC/CCL17 concentration in the sample to a reference TARC/CCL17 concentration value, wherein when the TARC/CCL17 concentration in the sample is greater than the reference TARC/CCL17 concentration value, the subject is identified as a candidate for a rheumatoid arthritis therapeutic regimen. In embodiments, the method can further comprising detecting at least one additional biomarker of rheumatoid arthritis in the sample.

In another embodiment, the disclosure provides a method for the diagnosis, prognosis and/or risk classification of a subject having or at risk of having rheumatoid arthritis, wherein the method comprises detecting an increased TARC/CCL17 concentration in the subject relative to a control subject not having rheumatoid arthritis.

In any of the methods, the TARC/CCL17 reference value can be the TARC/CCL17 concentration of a control sample or a TARC/CCL17 cutoff value. The TARC/CCL17 concentration can be, for example, the TARC/CCL17 plasma or serum concentration. The control sample can be a biological sample of a control subject or a TARC/CCL17 standard. The TARC/CCL17 concentration of a control sample can be, for example, the median TARC/CCL17 concentration of a plurality of control samples from a group of control subjects. Alternatively, a TARC/CCL17 cutoff value can be determined by a receiver operating curve (ROC) analysis from biological samples of a patient group. Alternatively, a TARC/CCL17 cutoff value can be determined by a quartile analysis of biological samples of a patient group. For example, a TARC/CCL17 cutoff value can be determined by selecting a value that corresponds to the median of a patient group consisting of patients with rheumatoid arthritis, which can be, for example, about 300-400 pg/ml serum. Alternatively, a TARC/CCL17 cutoff value can be determined by selecting a value that corresponds to the 75^{th} percentile of a patient group consisting of rheumatoid arthritis patients, which can be for example about 500 pg/mL serum. In other embodiments, an appropriate cutoff value can be about 70 pg/mL to about 2,500 pg/mL in serum. For example, a cutoff value of about 100-200 pg/mL serum may be used to discriminate rheumatoid arthritis specimens and normal specimens. Similar cutoff values can be used for blood plasma and blood samples.

In any of the methods, the subject can be a human subject and the biological sample of the subject and/or the control sample can be taken from a human subject. In any of the methods, the biological sample can be from a tissue or a bodily fluid, including any one of whole blood, plasma, serum, urine or any cell culture suspension or fraction of any thereof. In some embodiments of the methods described herein, the sample is whole blood, plasma, or serum, preferably plasma or serum. A coagulation inhibitor can be added to any peripheral blood sample. In the methods, determining the concentration of TARC/CCL17, and optionally the at least one additional biomarker, can be performed by an immunological assay method in which a reagent capable of specific binding to TARC/CCL17, and optionally a reagent capable of specific binding to the additional biomarker, are used.

In another aspect, the disclosure provides a kit for performing any of the methods and assays disclosed herein, wherein the kit includes at least one reagent capable of specifically binding TARC/CCL17, allowing for quantification of the TARC/CCL17 concentration in a biological sample from a subject, and a reference standard indicating a reference TARC/CCL17 concentration. In a kit for performing a method for providing a diagnosis, prognosis or risk classification of a subject having or at risk of having rheumatoid arthritis, the kit may further comprise at least one reagent capable of specifically binding at least one additional biomarker of rheumatoid arthritis in the biological sample, allowing for quantification of the concentration of the at least one additional biomarker in the biological sample, and a reference standard indicating a reference concentration of the at least one additional biomarker of rheumatoid arthritis in the biological sample. In any of the kits, the at least one reagent capable of specifically binding TARC/CCL17 may comprise at least one antibody capable of specifically binding TARC/CCL 17.

### BRIEF DESCRIPTION OF THE DRAWINGS

FIG. 1 depicts a scatter plot of individual samples taken from subjects having rheumatoid arthritis and not undergoing treatment, subjects having rheumatoid arthritis and undergoing treatment, or healthy control subjects. 95% CI (Confidence Interval) Mean Diamond in each of the categories is calculated by the measured values of specimens.

FIG. 2 is a Receiver Operating Characteristic (ROC) plot of the biomarkers Rheumatoid Factor (RF), C-reactive protein (CRP), matrix metalloproteinase-3 (MMP3), anti-cyclic citrullinated peptide antibody (Anti-CCP), and TARC/CCL17.

### DETAILED DESCRIPTION

The disclosure is based on the unexpected determination of a strong and independent association between thymus and activation-regulated chemokine (“TARC,” “CCL17,” or “TARC/CCL17” as used herein) and rheumatoid arthritis (RA). The disclosure identifies a novel association between increased (i.e., higher) TARC/CCL17 levels, and the incidence of RA and/or the likelihood of developing RA. As described herein, TARC/CCL17 is associated with RA. The association between TARC/CCL17 and RA is robust, predictive of disease onset, clinical progression, and/or disease severity in RA. Assessment of TARC/CCL17 can therefore improve on current methods and assays that are used to diagnose rheumatoid arthritis, provide prognosis of rheumatoid arthritis treatment or severity, and/or to stratify or identify patient risk of developing rheumatoid arthritis, thereby significantly benefiting patients having or at risk of developing rheumatoid arthritis. Further, combined use of TARC/CCL17 and additional biomarkers can provide comparable advantages.

Accordingly, the disclosure provides methods of providing a diagnosis, prognosis or risk classification/identification of a subject or group of subjects having or at risk of having rheumatoid arthritis, using TARC/CCL17 as a clinical biomarker. Also provided are methods of identification of a candidate subject or group of candidate subjects for a rheumatoid arthritis therapeutic regimen, where the methods utilize TARC/CCL17 as a biomarker. The disclosure also provides kits for performing the disclosed methods.

Section headings as used in this section and the entire disclosure herein are merely for organizational purposes and are not intended to be limiting.

### A. DEFINITIONS

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the terms “including” and “having,” as well as other forms of those terms, such as “includes,” “included”, “has,” and “have” are not limiting.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

“Control” as used herein when referring to a composition, refers to a composition known to not contain an analyte of interest (“negative”), e.g., TARC/CCL17 (such as TARC/CCL17, fragments of TARC/CCL17, variants of TARC/CCL17, or any combinations thereof); or to contain an analyte of interest (“positive control”), e.g., TARC/CCL17 (such as TARC/CCL17, fragments of TARC/CCL17, variants of TARC/CCL17, or any combinations thereof). A positive control can comprise a known concentration of TARC/CCL17. “Control,” “positive control,” and “calibrator” may be used interchangeably herein to refer to a composition comprising a known concentration of TARC/CCL17. A “positive control” can be used to establish assay performance characteristics and is a useful indicator of the integrity of reagents (e.g., analytes). A “normal control” or “healthy control” may refer to a sample or a subject that does not have rheumatoid arthritis, or is not at risk of developing rheumatoid arthritis.

As used herein, the term “thymus and activation-regulated chemokine,” “TARC,” “CCL17,” or “TARC/CCL17” are all interchangeable and refer to the Cys-Cys (CC) chemokine that is expressed constitutively in thymus and transiently in stimulated peripheral blood mononuclear cells. Other synonyms for TARC/CCL17 that have been used in the art include, Small-inducible cytokine A17, CC chemokine TARC, ABCD-2, SCYA17, A-152E5.3, MGC138271, and MGC138273. TARC/CCL17 can relate to any biological TARC/CCL17 sequence, including an amino acid sequence (e.g., protein, polypeptide, peptide (precursor or mature), fragments, truncations, fusions, derivatives, variants, etc.) and a nucleic acid sequence (e.g., DNA or RNA fragments, truncations, fusions, derivatives, SNPs, variants, etc). TARC/CCL17 can be from any organism and, in some embodiments, comprises a mammalian sequence such as, for example, a TARC/CCL17 selected from any of human (GenBank accession nos. NM_{—}002987, BC069107, BC112068, BC112066, D43767 (mRNA) and NP_{—}002978 (protein)), chimpanzee (P. troglodytes, GenBank XM_{—}523377 (mRNA) and XP_{—}523377 (protein)), monkey (M. mulatta, GenBank NM_{—}001032852 and AF449271 (mRNA) and NP_{—}001028024 (protein)), dog (C. familiaris, C. lupus, GenBank NM_{—}001003051 (mRNA) and NP_{—}001003051 (protein)), cow (B. taurus, GenBank XM_{—}001788943 (mRNA) and XP_{—}001788995 (protein)), mouse (M. musculus, GenBank NM_{—}011332 (mRNA) and NP_{—}035462 (protein)), and rat (R. norvegicus, GenBank NM_{—}057151 (mRNA) and NP_{—}476492 (protein).

In some embodiments TARC/CCL17 comprises human TARC/CCL17 (GenBank accession nos. NM_{—}002987 (mRNA) and NP_{—}002978 (protein)). In humans, the gene for TARC/CCL17 is located on the q arm of chromosome 16 (16q13), along with several other genes encoding CC cytokines. Human TARC/CCL17 cDNA encodes a 94 amino acid precursor protein that includes a signal peptide of 23 amino acids that is cleaved off to generate the mature secreted protein (71 amino acids). TARC/CCL17 is expressed in thymus, and at a lower level in the lung, colon, and small intestine. TARC/CCL17 is also transiently expressed in stimulated peripheral blood mononuclear cells. TARC/CCL17 is known to bind to chemokine receptors CCR4 and CCR8. TARC/CCL17 shows chemotactic activity for T lymphocytes, but not for monocytes or granulocytes. Functionally, TARC/CCL17 is involved in T cell development in thymus as well as in trafficking and activation of mature T cells. Elevated circulating TARC/CCL17 concentrations have been reported in plasma samples from patients with allergic diseases such as bronchial asthma and atopic dermatitis.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%, or at least about 40%, at least about 45%, at least about 50%, at least about 55%, at least about 60%, at least about 65%, at least about 70%, at least about 75%, at least about 80%, at least about 85%, at least about 90%, or at least about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., stage of disease, severity of disease, progression, non-progression, or improvement, etc.). The disclosure provides exemplary predetermined levels. However, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, the correlations as described herein should be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., TARC/CCL17, fragments of TARC/CCL17, variants of TARC/CCL17 or any combinations thereof) entails release (e.g., dissociation or decrease of binding) of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay. The pretreatment reagent optionally can comprise: (a) one or more solvents and salt, (b) one or more solvents, salt and detergent, (c) detergent, (d) detergent and salt, or (e) any reagent or combination of reagents appropriate for cell lysis and/or solubilization of analyte.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

“Sample,” “test sample,” “sample from a subject,” and “patient sample” may be used interchangeably herein. The sample, such as a sample of tissue, urine, serum, plasma, amniotic fluid, cerebrospinal fluid, placental cells or tissue, endothelial cells, leukocytes, or monocytes, can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Series of calibrating compositions” refers to a plurality of compositions comprising a known concentration of TARC/CCL17, wherein each of the compositions differs from the other compositions in the series by the concentration of TARC/CCL17.

“Specific binding partner” refers to a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Tracer” as used herein refers to an analyte or analyte fragment conjugated to a label, such as TARC/CCL17 conjugated to a fluorescein moiety, wherein the analyte conjugated to the label can effectively compete with the analyte for sites on an antibody specific for the analyte.

As used herein, the phrase “rheumatoid arthritis” or “RA” refers to a chronic, systemic autoimmune disease that occurs in about 1% of the population. While RA may affect many tissues and organs (e.g., about 15-25% of individuals with RA also develop anemia), it is typically characterized by chronic inflammation of the synovium, which often leads to progressive joint destruction and in most cases, to disability and reduction of quality of life. While onset of RA frequently occurs between the ages of 40 and 50, it can affect people of any age. As the pathology progresses, the inflammatory activity leads to tendon tethering and erosion and destruction of the joint surface, which impairs range of motion/movement and leads to joint deformity.

A diagnosis of RA is typically made by any one or more clinical or diagnostic tests including, for example, imaging techniques (e.g., X-rays, magnetic resonance imaging (MRI) and ultrasound (e.g., color Doppler and power Doppler ultrasound); blood/lab tests (e.g., erythrocyte sedimentation rate (ESR), full blood count, renal function, liver enzymes, immunological analysis using rheumatoid factor (RF), anti-citrullinated protein/peptide antibodies (ACPAs)/anti-cyclic citrullinated protein antibodies (anti-CCP), anti-mutated citrullinated vimentin antibody (anti-MCV) assay, matrix metalloproteinase-3 (MMP3), C-reactive protein (CRP), and antinuclear antibody (ANA)).

The American College of Rheumatology (ACR) and the European League Against Rheumatism (EULAR) have published diagnostic guidelines. These organizations together recently published guidelines that are adapted for early RA diagnosis 2010 ACR/EULAR Rheumatoid Arthritis Classification Criteria (Aletaha D, Neogi T, Silman A J, et al. (September 2010). “2010 rheumatoid arthritis classification criteria: an American College of Rheumatology/European League Against Rheumatism collaborative initiative”. Ann. Rheum. Dis. 69 (9): 1580-8, incorporated herein by reference.) Briefly, the classification criteria establish a point value between 0 and 10. Any patient having a point total of 6 or more is classified as an RA patient, provided the patient has synovitis in at least one joint and no other diagnosis can justify the cause of synovitis. The diagnosis guidelines provide four areas of coverage: (i) joint involvement (e.g., number/size of joints involved); (ii) serological parameters (e.g., positive RF and ACPA (and amounts thereof)); (iii) acute phase reactants (e.g., elevated erythrocyte sedimentation rate (ESR) and/or elevated CRP values); and (iv) disease duration (1 point for symptoms lasting six weeks or longer). In these criteria serology and autoimmune diagnostics carries major weight, as such tests have been able to improve early stage disease (e.g., ACPA detection).

A routine clinical diagnosis of RA is commonly made based on any one or more of patient symptoms (e.g., morning stiffness lasting from over one hour for up to six weeks), physical examination (e.g., swollen joints (e.g., two or more)), radiographs (X-rays), and lab diagnostics as described herein or as known in the art. For example, in addition to immunological tests mentioned above (e.g., RF and ACPA), blood tests can be performed at early diagnosis stage to analyze ESR, CRP, full blood count, renal function, liver enzymes and other immunological tests (e.g., antinuclear antibody (ANA)). Point-of-care assays can also be used to detect serological markers including RF, ACPA and MCV).

While there is no known cure for rheumatoid arthritis, many treatments can alleviate symptoms and/or modify the disease process. Currently, early and aggressive treatments have shown an increase in therapeutic efficacy, including remission or substantial alleviation of symptoms for increasing percentages of patients. Treatment encompasses two goals, (i) alleviating symptoms, and (ii) preventing joint damage/destruction. A variety of RA treatments are known in the art. Non-pharmacological treatment options can include physical therapy, orthoses, occupational therapy and nutritional therapy but these therapies do not stop progression of joint destruction. Pharmaceuticals such as analgesics and anti-inflammatory drugs (e.g., NSAIDS), including steroids, are often used to suppress the symptoms, while disease-modifying antirheumatic drugs (DMARDs) are administered to slow or stop the underlying immune response and prevent long-term joint damage. A number of biologic therapeutics are approved for RA treatment in adults. Non-limiting examples of all such therapies include analgesics (e.g., acetaminophen and tramadol); narcotics (e.g., propoxyphene, propoxyphene/acetaminophen, oxycodone, hydrocodone/acetaminophen); NSAIDs (e.g., celecoxib, diclofenac sodium, ibuprofen, naproxen, ketorolac); steroids (e.g., prednisone); non-biologic DMARDs, (e.g., auranofin (oral gold), azathioprine, cyclosporine, gold sodium thiomalate (injectable gold), hydroxychloroquine sulfate, leflunomide, methotrexate, minocycline, sulfasalazine); and biologic DMARDs (e.g., abatacept (Orencia®), adalimumab (Humira®), anakinra (Kineret®), certolizumab pegol (Cimzia®), etanercept (Enbrel®), golimumab (Simponi®), infliximab (Remicade®), rituximab (Rituxan®), tocilizumab (Actemra®).

As is known in the art, RA can be associated with, cause, or contribute to any number of other diseases or disorders. Thus, in embodiments of the methods that relate to identification of a patient population for RA therapy, the methods provide for treating or preventing conditions that are associated with or caused by RA in a patient. Such conditions can include disorders of the skin (e.g., rheumatoid nodule, vasculitis, neutrophilic dermatoses (e.g., pyoderma gangrenosum, Sweet's syndrome), erythema nodosum, lobular panniculitis, atrophy of digital skin, palmar erythema, diffuse thinning (rice paper skin), and skin fragility); lung (e.g., Rheumatoid Lung Disease, pleural effusions and lung fibrosis); kidneys (e.g., renal amyloidosis and membranous nephropathy); heart and blood vessels (e.g., atherosclerosis, increased risk of heart attack and stroke, pericarditis, endocarditis, left ventricular failure, valvulitis and fibrosis); eyes (e.g., keratoconjunctivitis sicca, episcleritis, perforating scleromalacia, and keratitis); blood and liver disorders (e.g., anemia, thrombocytosis, neutropenia, Felty's syndrome, nodular hyperplasia, and hepatitis); swelling of the spleen; neurological disorders (e.g., carpal tunnel syndrome, atlanto-axial subluxation, spinal cord compression, peripheral neuropathy and mononeuritis multiplex); osteoporosis, and some cancers (e.g., lymphoma).

As used herein, the terms “risk assessment,” “risk classification,” “risk identification,” or “risk stratification” of subjects (e.g., patients) refers to the evaluation of factors including biomarkers, to predict the risk of occurrence of future events including disease onset or disease progression, so that treatment decisions regarding the subject may be made on a more informed basis.

As used herein, the term “RA risk” or “rheumatoid arthritis risk” of a subject refers to the evaluation of factors including biomarkers, to predict the risk of occurrence of RA including increased probability of RA onset, RA progression, and occurrence/severity of clinical symptoms associated with RA. While the course of RA can vary, with some patients having mild short-term symptoms, the majority of RA patients have disease that is progressive for life. In addition to TARC/CCL17 levels, other factors that may indicate a poor prognosis include persistent synovitis, early erosive disease, extra-articular findings (e.g., subcutaneous rheumatoid nodules), seropositive RF, seropositive anti-CCP (e.g., MCV), genetic factors (e.g., having HLA-DR4 “Shared Epitope” alleles), family history of RA, poor functional status, socioeconomic factors, elevated acute phase response (ESR, CRP), and increased clinical severity. Thus, in some embodiments, the method relate to providing a prognosis of RA onset or RA progression comprising detecting/determining the level of TARC/CCL17 in a sample from a patient, in combination with any one or more prognostic factors described herein, or known in the art.

As used herein, the terms “specific binding” or “specifically binding”, refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present disclosure include, but are not limited to, antiserum, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. In some embodiments, the subject may be a human patient at risk for developing, or already having, rheumatoid arthritis.

The terms “sample” and “biological sample” as used herein generally refer to a biological material being tested for and/or suspected of containing an analyte of interest such as TARC/CCL17. The sample may be any tissue sample taken or derived from the subject. In some embodiments, the sample from the subject may comprise protein. In some embodiments, the sample from the subject may comprise nucleic acid (e.g., polynucleotide, mRNA, etc.).

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, bronchoalveolar lavage (BAL) fluid, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing urine, blood, serum and plasma, and other body fluids, are used in the practice of the present disclosure, for instance, when the antibodies provided herein are employed as immunodiagnostic reagents, and/or in TARC/CCL17 immunoassay kit. The test sample can comprise further moieties in addition to the TARC/CCL17 analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

The pretreatment reagent can be any reagent appropriate for use with the assay, e.g., immunoassay, and kit described herein. The pretreatment optionally comprises: (a) one or more solvents (e.g., methanol and ethylene glycol) and salt, (b) one or more solvents, salt and detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., Abbott TDx Monoclonal Antibody Assay Evaluated for Measuring Cyclosporine in Whole Blood, Clin. Chem. 36: 1969-1973 (1990), and Wallemacq et al., Evaluation of the New AxSYM Cyclosporine Assay: Comparison with TDx Monoclonal Whole Blood and EMIT Cyclosporine Assays, Clin. Chem. 45: 432-435 (1999)), and/or as commercially available. Additionally, pretreatment can be done as described in Abbott's U.S. Pat. No. 5,135,875, European Pat. Pub. No. 0 471 293, and U.S. Pat. App. Pub. No. 2008/0020401 (incorporated by reference in its entirety for its teachings regarding pretreatment). The pretreatment reagent can be a heterogeneous agent or a homogeneous agent.

With use of a heterogeneous pretreatment reagent, the pretreatment reagent precipitates analyte binding protein (e.g., protein that can bind to TARC/CCL17 or a fragment thereof) present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of test sample and pretreatment reagent are contacted with a labeled specific binding partner for TARC/CCL17, variants of TARC/CCL17 such as a labeled anti-TARC/CCL17 monoclonal antibody (or an antigenically reactive fragment thereof). The pretreatment reagent employed for such an assay typically is diluted in the pretreated test sample mixture, either before or during capture by the first specific binding partner. Despite such dilution, a certain amount of the pretreatment reagent (for example, 5 M methanol and/or 0.6 M ethylene glycol) is still present (or remains) in the test sample mixture during capture.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### B. METHODS

The methods encompass providing a diagnosis or a prognosis of a subject which includes, with respect to RA, any one or more of determining the that the subject has RA, determining the severity of RA, determining the subject's risk for developing RA (i.e., likelihood of disease onset), determining the efficacy of a RA treatment regimen, identifying a subject as a candidate for RA therapy, and risk assessment regarding progression of RA in a subject having the disease. The methods are based in part on the unexpected finding that TARC/CCL17 concentration in a biological sample from a subject is predictive or diagnostic of rheumatoid arthritis in the subject, and thus TARC/CCL17 can be used as a prognostic or diagnostic biomarker for rheumatoid arthritis.

The methods involve providing or obtaining a biological sample from the subject, which can be obtained by any known means including needle stick, needle biopsy, swab, and the like. In an embodiment of the method, the biological sample is a blood sample, preferably a blood plasma or serum sample, which may be obtained by any standard technique such as, for example, by venipuncture. Biological samples used in the methods may be stored or banked under suitable tissue storage conditions, or can be accessed from samples that have been previously stored or banked under suitable conditions. In some embodiments, the methods comprise reviewing data from a prior assay or analysis of a biological sample from the subject (e.g., measurement of TARC/CCL17 and/or another RA biomarker such as, for example, any one or more of RF, CRP, MMP3, and anti-CCP).

The methods encompass a method for diagnosis, prognosis and/or risk stratification of RA in a subject having or suspected of having RA by determining TARC/CCL17 concentration in the subject. Providing a diagnosis can be, for example, providing a diagnosis of RA in a subject, where the subject can be previously undiagnosed as having with RA, (or not identified as having a risk at risk of having RA), suspected of having RA, or not. Alternatively, or in addition, providing a prognosis can be, for example, determining RA severity, or can be a risk assessment, i.e. determination of the likelihood that the subject will develop RA. The methods also encompass identifying one or more patients or a subgroup of patients having an increased risk of developing RA. A shared feature of all methods is the determination of concentration of TARC/CCL17 in a biological sample as described herein, wherein an increased concentration of TARC/CCL17 in the sample relative to a reference value for TARC/CCL17 concentration is indicative of RA, or increased risk of developing RA.

The TARC/CCL17 concentration is deemed increased in comparison to a reference value or predetermined level, i.e., the reference TARC/CCL17 concentration value as described herein. For example, an TARC/CCL17 serum concentration useful as a reference concentration value is about 32 pg/ml, but can be higher or lower, for example about 20 pg/ml or about 100 pg/ml in serum (e.g., about 20, 25, 30, 35, 40, 45, 50, 55, 60, 65, 70, 75, 80, 85, 90, 95 or 100 pg/ml). The TARC/CCL17 concentration may be deemed increased as compared to the reference value when it is detectably higher (e.g., from about 1% to about 10% higher), or significantly higher, e.g. at least 20% higher (1.2 fold), at least 30% (1.3 fold) higher, at least 40% higher (1.4 fold), at least 50% higher (1.5 fold), at least 60% higher (1.6 fold), at least 70% higher (1.7 fold), at least 80% higher (1.8 fold), at least 100% higher (2.0 fold or double), at least 150% higher (2.5 fold), or at least 200% higher (3.0 fold or triple).

The presence, concentration or amount of TARC/CCL17 in a biological sample may be readily determined using any suitable assay as is known in the art. Examples include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay, a capture reagent that specifically binds TARC/CCL17 (or a fragment thereof) of interest is attached to the surface of a mass spectrometry probe, such as a pre-activated protein chip array. The TARC/CCL17 (including fragments of TARC/CCL17, variants of TARC/CCL17, or any combinations thereof) is then specifically captured on the biochip, and the captured TARC/CCL17 is detected by mass spectrometry. Alternatively, the TARC/CCL17 can be eluted from the capture reagent and detected by traditional MALDI (matrix-assisted laser desorption/ionization) or by SELDI. A chemiluminescent microparticle immunoassay, in particular one employing the ARCHITECT® automated analyzer (Abbott Laboratories, Abbott Park, Ill.), is an example of a preferred immunoassay. Other methods include, for example, mass spectrometry and immunohistochemistry (e.g. with sections from tissue biopsies) using antibodies (monoclonal, polyclonal, chimeric, humanized, human, etc.) or fragments thereof that specifically bind TARC/CCL17. Anti-TARC/CCL17 antibodies and fragments thereof can be produced according to methods known in the art as described herein. Alternatively, commercially available anti-TARC/CCL17 antibodies can be used as described herein. Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety.

TARC/CCL17, and/or fragments, or variants, or any combinations thereof, may be analyzed using an immunoassay. The presence or amount of TARC/CCL17 can be determined using antibodies and detecting specific binding to TARC/CCL17. If desired, one or more of the antibodies described herein can be used in combination with one or more commercially available monoclonal/polyclonal antibodies. Such antibodies are commercially available from companies such as Novus Biologicals (Littleton, Colo.), Cell Sciences (Canton, Mass.), US Biological (Swampscott, Mass.), Santa Cruz Biotechnology (Santa Cruz, Calif.), AbD Serotec (Raleigh, N.C.), R&D Systems, Inc. (Minneapolis, Minn.), LifeSpan Biosciences (Seattle, Wash.), Thermo Scientific Pierce Products (Rockford, Ill.), GeneTex (Irvine, Calif.), Abnova (Taiwan & Walnut, Calif.), Sigma-Aldrich (St. Louis, Mo.), and Enzo Life Sciences International, Inc. (Plymouth Meeting, Pa.).

Any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA.

A heterogeneous format may be used. For example, after the test sample is obtained from a subject, a first mixture is prepared. The mixture contains the test sample being assessed for TARC/CCL17 (including fragments of TARC/CCL17, variants of TARC/CCL17, or any combinations thereof) and a first specific binding partner, wherein the first specific binding partner and any TARC/CCL17 contained in the test sample form a first specific binding partner-TARC/CCL17 complex. Preferably, the first specific binding partner is an anti-TARC/CCL17 antibody or a fragment thereof. The order in which the test sample and the first specific binding partner are added to form the mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first specific binding partner-TARC/CCL17 complex is formed, any unbound TARC/CCL17 is removed from the complex using any technique known in the art. For example, the unbound TARC/CCL17 can be removed by washing. Suitably, however, the first specific binding partner is present in excess of any TARC/CCL17 present in the test sample, such that all TARC/CCL17 that is present in the test sample is bound by the first specific binding partner.

After any unbound TARC/CCL17 is removed, a second specific binding partner is added to the mixture to form a first specific binding partner—TARC/CCL17-second specific binding partner complex. The second specific binding partner is preferably an anti-TARC/CCL17 antibody that binds to an epitope on TARC/CCL17 that differs from the epitope on TARC/CCL17 bound by the first specific binding partner. Moreover, also preferably, the second specific binding partner is labeled with or contains a detectable label as described above.

As noted above, use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

The Sandwich ELISA measures the amount of antigen between two layers of antibodies (i.e., a capture antibody (i.e., at least one capture antibody) and a detection antibody (i.e. at least one detection antibody). The capture antibody and the detection antibody bind to different epitopes on the antigen, e.g., TARC/CCL17. Desirably, binding of the capture antibody to an epitope does not interfere with binding of the detection antibody to an epitope. Either monoclonal or polyclonal antibodies may be used as the capture and detection antibodies in the sandwich ELISA.

Generally, at least two antibodies are employed to separate and quantify TARC/CCL17 (inclusive of TARC/CCL17, fragments of TARC/CCL17, variants of TARC/CCL17, or any combinations thereof) in a test sample. More specifically, the at least two antibodies bind to certain epitopes of TARC/CCL17 or a TARC/CCL17 fragment forming an immune complex which is referred to as a “sandwich”. One or more antibodies can be used to capture the TARC/CCL17 (e.g., TARC/CCL17, fragments of TARC/CCL17, variants of TARC/CCL17, or any combinations thereof) in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. In other words, antibodies are selected so that the one or more first antibodies brought into contact with a test sample containing, or suspected of containing TARC/CCL17 (e.g., TARC/CCL17, fragments of TARC/CCL17, variants of TARC/CCL17, or any combinations thereof) do not bind to all or part of an epitope recognized by the second or subsequent antibodies, thereby not interfering with the ability of the one or more second detection antibodies to bind to the TARC/CCL17 (e.g., TARC/CCL17, fragments of TARC/CCL17, variants of TARC/CCL17, or any combinations thereof).

The antibodies may be used as a first antibody in said immunoassay. Preferably, the antibody immunospecifically binds to an epitope comprising at least three contiguous (3) amino acids of TARC/CCL17 with a K_{D} of from 4.2×10⁻¹¹ M to 7.4×10⁻¹³ M. The immunoassay may comprise a second antibody that immunospecifically binds to an epitope comprising at least three contiguous (3) amino acids of TARC/CCL17, wherein the contiguous (3) amino acids to which the second antibody binds is different from the three (3) contiguous amino acids to which the first antibody binds.

In an embodiment, a test sample suspected of containing TARC/CCL17 (e.g., TARC/CCL17, fragments of TARC/CCL17, variants of TARC/CCL17, or any combinations thereof) can be contacted with at least one capture antibody (or antibodies) and at least one detection antibodies either simultaneously or sequentially. In the sandwich assay format, a test sample suspected of containing TARC/CCL17 (e.g., TARC/CCL17, fragments of TARC/CCL17, variants of TARC/CCL17, or any combinations thereof) is first brought into contact with the at least one capture antibody that specifically binds to a particular epitope under conditions which allow the formation of a antibody-TARC/CCL17 complex. If more than one capture antibody is used, a multiple capture antibody-TARC/CCL17 complex is formed. In a sandwich assay, the antibodies, preferably, the at least one capture antibody, are used in molar excess amounts of the maximum amount of TARC/CCL17 or the TARC/CCL17 fragment expected in the test sample. For example, from about 5 μg/mL to about 1 mg/mL of antibody per mL of microparticle coating buffer may be used.

Optionally, prior to contacting the test sample with the at least one first capture antibody, the at least one capture antibody can be bound to a solid support which facilitates the separation the antibody-TARC/CCL17 complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind TARC/CCL17 or TARC/CCL17 fragment. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample suspected of containing TARC/CCL17 (e.g., TARC/CCL17, fragments of TARC/CCL17, variants of TARC/CCL17, or any combinations thereof) is brought into contact with the at least one capture antibody, the test sample is incubated in order to allow for the formation of a capture antibody (or capture antibodies)-TARC/CCL17 complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the capture antibody (antibodies)-TARC/CCL17 complex, the complex is then contacted with at least one detection antibody (under conditions which allow for the formation of a capture antibody (antibodies)-TARC/CCL17-detection antibody (antibodies) complex). If the capture antibody-TARC/CCL17 complex is contacted with more than one detection antibody, then a capture antibody (antibodies)-TARC/CCL17-detection antibody (antibodies) detection complex is formed. As with the capture antibody, when the at least one detection (and subsequent) antibody is brought into contact with the capture antibody-TARC/CCL17 complex, a period of incubation under conditions similar to those described above is required for the formation of the capture antibody (antibodies)-TARC/CCL17-detection antibody (antibodies) complex. Preferably, at least one detection antibody contains a detectable label. The detectable label can be bound to the at least one detection antibody prior to, simultaneously with or after the formation of the capture antibody (antibodies)-TARC/CCL17-detection antibody (antibodies) complex. Any detectable label known in the art can be used as discussed herein and known in the art.

Chemiluminescent assays can be performed in accordance with the methods described in Adamczyk et al., Anal. Chim. Acta 579(1): 61-67 (2006). While any suitable assay format can be used, a microplate chemiluminometer (Mithras LB-940, Berthold Technologies U.S.A., LLC, Oak Ridge, Tenn.) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-TARC/CCL17 complexes form. Alternatively, if a second specific binding partner is used and the second specific binding partner is detectably labeled with an acridinium compound, detectably labeled first specific binding partner-TARC/CCL17-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of TARC/CCL17 or a fragment thereof is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of TARC/CCL17 (e.g., TARC/CCL17, fragments of TARC/CCL17, variants of TARC/CCL17, or any combinations thereof) in the sample can be quantified. Specifically, the amount of TARC/CCL17 in the sample is proportional to the intensity of the signal generated. The amount of TARC/CCL17 present can be quantified by comparing the amount of light generated to a standard curve for TARC/CCL17 or by comparison to a reference standard. The standard curve can be generated using serial dilutions or solutions of known concentrations of TARC/CCL17 by mass spectroscopy, gravimetric methods, and other techniques known in the art.

In a chemiluminescent microparticle assay employing the ARCHITECT® (or its successor) analyzer, the conjugate diluent pH should be about 5.8+/−0.2, the microparticle coating buffer should be maintained at room temperature (i.e., at about 17 to about 27° C.), the microparticle coating buffer pH should be about 5.5+/−0.2, and the microparticle diluent pH should be about 6.0+/−0.2. Solids preferably are less than about 0.2%, such as less than about 0.15%, less than about 0.14%, less than about 0.13%, less than about 0.12%, less than about 0.11%, less than about 0.10%, less than about 0.09%, less than about 0.08%, less than about 0.07%, less than about 0.06%, less than about 0.05%, less than about 0.04%, or less than about 0.03%, such as about 0.025%.

In a forward competitive format, an aliquot of labeled TARC/CCL17 (e.g., TARC/CCL17, fragments of TARC/CCL17, variants of TARC/CCL17, or any combinations thereof) of a known concentration is used to compete with TARC/CCL17 in a test sample for binding to TARC/CCL17 antibody (such as an immobilized TARC/CCL17 antibody).

In a forward competition assay, an immobilized antibody (such as a TARC/CCL17 antibody) can either be sequentially or simultaneously contacted with the test sample and a labeled TARC/CCL17, TARC/CCL17 fragment, or TARC/CCL17 variant. The TARC/CCL17 protein, TARC/CCL17 fragment or TARC/CCL17 variant can be labeled with any detectable label, including those detectable labels discussed above in connection with the anti-TARC/CCL17 antibodies. In this assay, the antibody can be immobilized on to a solid support. Alternatively, the antibody can be coupled to an antibody, such as an anti-species antibody, that has been immobilized on a solid support, such as a microparticle.

The labeled TARC/CCL17 (e.g., TARC/CCL17, fragments of TARC/CCL17, variants of TARC/CCL17, or any combinations thereof), the test sample, and the antibody are incubated under conditions similar to those described above in connection with the sandwich assay format. Two different species of antibody-TARC/CCL17 complexes may then be generated. Specifically, one of the antibody-TARC/CCL17 complexes generated contains a detectable label while the other antibody-TARC/CCL17 complex does not contain a detectable label. The antibody-TARC/CCL17 complex can be, but does not have to be, separated from the remainder of the test sample prior to quantification of the detectable label. Regardless of whether the antibody-TARC/CCL17 complex is separated from the remainder of the test sample, the amount of detectable label in the antibody-TARC/CCL17 complex is then quantified. The concentration of TARC/CCL17 (e.g., TARC/CCL17, fragments of TARC/CCL17, variants of TARC/CCL17, or any combinations thereof) in the test sample can then be determined by comparing the quantity of detectable label in the antibody-TARC/CCL17 complex to a standard curve. The standard curve can be generated using serial dilutions of TARC/CCL17 (e.g., TARC/CCL17, fragments of TARC/CCL17, variants of TARC/CCL17, or any combinations thereof) of known concentration, by mass spectroscopy, gravimetrically and by other techniques known in the art.

The antibody-TARC/CCL17 complex can be separated from the test sample by binding the antibody to a solid support, such as the solid supports discussed above in connection with the sandwich assay format, and then removing the remainder of the test sample from contact with the solid support.

In a reverse competition assay, an immobilized TARC/CCL17 (e.g., TARC/CCL17, fragments of TARC/CCL17, variants of TARC/CCL17, or any combinations thereof) can either be sequentially or simultaneously contacted with a test sample and at least one labeled antibody. Preferably, the antibody specifically binds to an epitope comprising at least 3, at least 4, at least 5, at least 6, at least 7, at least 8, at least 9, or at least 10, at least 15, at least 20, at least 25 or at least 30 amino acids of TARC/CCL17.

The TARC/CCL17 (e.g., TARC/CCL17, fragments of TARC/CCL17, variants of TARC/CCL17, or any combinations thereof) can be bound to a solid support, such as the solid supports discussed above in connection with the sandwich assay format. Preferably, the TARC/CCL17 fragment comprises at least 3, at least 4, at least 5, at least 6, at least 7, at least 8, at least 9, at least 10, at least 15, at least 20, at least 25 or at least 30 amino acids of the TARC/CCL17 protein.

The immobilized TARC/CCL17 (e.g., TARC/CCL17, fragments of TARC/CCL17, variants of TARC/CCL17, or any combinations thereof), test sample, and at least one labeled antibody are incubated under conditions similar to those described above in connection with the sandwich assay format. Two different species of TARC/CCL17-antibody complexes are then generated. Specifically, one of the TARC/CCL17-antibody complexes generated is immobilized and contains a detectable label while the other TARC/CCL17-antibody complex is not immobilized and contains a detectable label. The non-immobilized TARC/CCL17-antibody complex and the remainder of the test sample are removed from the presence of the immobilized TARC/CCL17-antibody complex through techniques known in the art, such as washing. Once the non-immobilized TARC/CCL17 antibody complex is removed, the amount of detectable label in the immobilized TARC/CCL17-antibody complex is then quantified. The concentration of TARC/CCL17 (e.g., TARC/CCL17, fragments of TARC/CCL17, variants of TARC/CCL17, or any combinations thereof) in the test sample can then be determined by comparing the quantity of detectable label in the TARC/CCL17-complex to a standard curve. The standard curve can be generated using serial dilutions of TARC/CCL17 or TARC/CCL17 fragment of known concentration, by mass spectroscopy, gravimetrically and by other techniques known in the art.

In a fluorescence polarization assay, an antibody or functionally active fragment thereof may be first contacted with an unlabeled test sample suspected of containing TARC/CCL17 (e.g., TARC/CCL17, fragments of TARC/CCL17, variants of TARC/CCL17, or any combinations thereof) to form an unlabeled TARC/CCL17-antibody complex. The unlabeled TARC/CCL17-antibody complex is then contacted with a fluorescently labeled TARC/CCL17 (e.g., TARC/CCL17, fragments of TARC/CCL17, variants of TARC/CCL17, or any combinations thereof). The labeled TARC/CCL17 competes with any unlabeled TARC/CCL17 in the test sample for binding to the antibody or functionally active fragment thereof. The amount of labeled TARC/CCL17-antibody complex formed is determined and the amount of TARC/CCL17 (e.g., TARC/CCL17, fragments of TARC/CCL17, variants of TARC/CCL17, or any combinations thereof) in the test sample determined via use of a standard curve.

The antibody used in a fluorescence polarization assay specifically binds to an epitope comprising at least 3, at least 4, at least 5, at least 6, at least 7, at least 8, at least 9, at least 10, at least 15, at least 20, at least 25 or at least 30 amino acids of TARC/CCL17.

The antibody, labeled TARC/CCL17 (e.g., TARC/CCL17, fragments of TARC/CCL17, variants of TARC/CCL17, or any combinations thereof), test sample and at least one labeled antibody may be incubated under conditions similar to those described above in connection with the sandwich immunoassay.

Alternatively, an antibody or functionally active fragment thereof may be simultaneously contacted with a fluorescently labeled TARC/CCL17 (e.g., TARC/CCL17, fragments of TARC/CCL17, variants of TARC/CCL17, or any combinations thereof) and an unlabeled test sample suspected of containing TARC/CCL17 (e.g., TARC/CCL17, fragments of TARC/CCL17, variants of TARC/CCL17, or any combinations thereof) to form both labeled TARC/CCL17-antibody complexes and unlabeled TARC/CCL17-antibody complexes. The amount of labeled TARC/CCL17-antibody complex formed is determined and the amount of TARC/CCL17 in the test sample determined via use of a standard curve. The antibody used in this immunoassay specifically may bind to an epitope comprising at least 3, at least 4, at least 5, at least 6, at least 7, at least 8, at least 9, at least 10, at least 15, at least 20, at least 25 or at least 30 amino acids of TARC/CCL17.

Alternatively, an antibody or functionally active fragment thereof is first contacted with a fluorescently labeled TARC/CCL17 (e.g., TARC/CCL17, fragments of TARC/CCL17, variants of TARC/CCL17, or any combinations thereof) to form a labeled TARC/CCL17-antibody complex. The labeled TARC/CCL17-antibody complex is then contacted with an unlabeled test sample suspected of containing TARC/CCL17 (e.g., TARC/CCL17, fragments of TARC/CCL17, variants of TARC/CCL17, or any combinations thereof). Any unlabeled TARC/CCL17 in the test sample competes with the TARC/CCL17 (e.g., TARC/CCL17, fragments of TARC/CCL17, variants of TARC/CCL17, or any combinations thereof) for binding to the antibody or functionally active fragment thereof. The amount of labeled TARC/CCL17-antibody complex formed is used to determine the amount of TARC/CCL17 in the test sample via use of a standard curve. The antibody used in this immunoassay specifically binds to an epitope comprising at least three 3, at least 4, at least 5, at least 6, at least 7, at least 8, at least 9, at least 10, at least 15, at least 20, at least 25, or at least 30 amino acids of TARC/CCL17.

Mass spectrometry (MS) analysis may be used alone or in combination with other methods. Other methods include immunoassays and those described above to detect specific polynucleotides. The mass spectrometry method may be used to determine the presence and/or quantity of one or more biomarkers. MS analysis may comprise matrix-assisted laser desorption/ionization (MALDI) time-of-flight (TOF) MS analysis, such as, for example, directed-spot MALDI-TOF or liquid chromatography MALDI-TOF analysis. In some embodiments, the MS analysis comprises electrospray ionization (ESI) MS, such as liquid chromatography (LC) ESI-MS. Mass analysis can be accomplished using commercially available spectrometers. Methods for utilizing MS analysis, including MALDI-TOF MS and ESI-MS, to detect the presence and quantity of biomarker peptides in biological samples may be used. See, for example, U.S. Pat. Nos. 6,925,389; 6,989,100; and 6,890,763 for guidance, each of which is incorporated herein by reference.

It may be desirable to include a control sample or a calibrator, such as a series of calibrators. The control sample may be analyzed concurrently with the sample from the subject as described above. The results obtained from the subject sample can be compared to the results obtained from the control sample. Standard curves may be provided, with which assay results for the biological sample may be compared. Such standard curves present levels as a function of assay units, i.e., fluorescent signal intensity, if a fluorescent label is used. Using samples taken from multiple donors, standard curves can be provided for control levels of the TARC/CCL17 in normal tissue, as well as for “at-risk” levels of the TARC/CCL17 in tissue taken from donors, who may have one or more of the characteristics set forth above.

Thus, in view of the above, a method of determining the presence, amount or concentration of TARC/CCL17 (e.g., TARC/CCL17, fragments of TARC/CCL17, variants of TARC/CCL17, or any combinations thereof) in a test sample is provided. The method comprises assaying the test sample for TARC/CCL17 (e.g., TARC/CCL17, fragments of TARC/CCL17, variants of TARC/CCL17, or any combinations thereof) by an immunoassay, for example, employing at least one antibody and at least one detectable label and comprising comparing a signal generated by the detectable label as a direct or indirect indication of the presence, amount or concentration of TARC/CCL17 in the test sample to a signal generated as a direct or indirect indication of the presence, amount or concentration of TARC/CCL17 in a calibrator. The calibrator is optionally, and is preferably, part of a series of calibrators in which each of the calibrators differs from the other calibrators in the series by the concentration of TARC/CCL17. One of the at least one antibody is an isolated antibody, which specifically binds to TARC/CCL17 (e.g., TARC/CCL17, fragments of TARC/CCL17, variants of TARC/CCL17, or any combinations thereof), wherein the antibody has a domain or region selected from (i) a variably heavy domain region, or (ii) a variably heavy domain region and a variable light domain region. Alternatively, one of the at least one antibody is an isolated antibody, which specifically binds to TARC/CCL17 (e.g., TARC/CCL17, fragments of TARC/CCL17, variants of TARC/CCL17, or any combinations thereof), wherein the antibody has (i) a variable heavy chain comprising a complementarity determining region (CDR)1, a CDR2, and a CDR3 and a variable light chain comprising a CDR1, a CDR2, and a CDR3. An example of at least one antibody that can be used are antibodies which specifically bind to TARC/CCL17 such as those commercially available from companies such as Novus Biologicals (Littleton, Colo.), Cell Sciences (Canton, Mass.), US Biological (Swampscott, Mass.), Santa Cruz Biotechnology (Santa Cruz, Calif.), AbD Serotec (Raleigh, N.C.), R&D Systems, Inc. (Minneapolis, Minn.), LifeSpan Biosciences (Seattle, Wash.), Thermo Scientific Pierce Products (Rockford, Ill.), GeneTex (Irvine, Calif.), Abnova (Taiwan & Walnut, Calif.), Sigma-Aldrich (St. Louis, Mo.), and Enzo Life Sciences International, Inc. (Plymouth Meeting, Pa.).

The method can comprise (i) contacting the test sample with at least one capture antibody, which binds to an epitope on TARC/CCL17 (e.g., TARC/CCL17, fragments of TARC/CCL17, variants of TARC/CCL17, or any combinations thereof), so as to form a capture antibody/TARC/CCL17 complex, (ii) contacting the capture antibody/TARC/CCL17 complex with at least one detection antibody, which comprises a detectable label and binds to an epitope on TARC/CCL17 (e.g., TARC/CCL17, fragments of TARC/CCL17, variants of TARC/CCL17, or any combinations thereof) that is not bound by the capture antibody, to form a capture antibody/TARC/CCL17/detection antibody complex, and (iii) determining the amount of TARC/CCL17 in the test sample based on the signal generated by the detectable label in the capture antibody/TARC/CCL17/detection antibody complex formed in (ii).

Alternatively, the method can comprise (i) contacting the test sample with at least one capture antibody, which binds to an epitope of TARC/CCL17 (e.g., TARC/CCL17, fragments of TARC/CCL17, variants of TARC/CCL17, or any combinations thereof) so as to form a capture antibody/TARC/CCL17 complex, and simultaneously or sequentially, in either order, contacting the test sample with detectably labeled TARC/CCL17, which can compete with any TARC/CCL17 (e.g., TARC/CCL17, fragments of TARC/CCL17, variants of TARC/CCL17, or any combinations thereof) in the test sample for binding to the at least one capture antibody. Any TARC/CCL17 (e.g., TARC/CCL17, fragments of TARC/CCL17, variants of TARC/CCL17, or any combinations thereof) present in the test sample and the detectably labeled TARC/CCL17 compete with each other to form a capture antibody/TARC/CCL17 complex and a capture antibody/detectably labeled TARC/CCL17 complex, respectively. The method further comprises (ii) determining the presence, amount or concentration of TARC/CCL17 in the test sample based on the signal generated by the detectable label in the capture antibody/detectably labeled TARC/CCL17 complex formed in (ii). The signal generated by the detectable label in the capture antibody/detectably labeled TARC/CCL17 complex is inversely proportional to the amount or concentration of TARC/CCL17 in the test sample.

In some embodiments, the methods can comprise any techniques and assays that are used in the art to measure the amount of TARC/CCL17 in a sample. For example, a polyclonal, monoclonal, chimeric, humanized or human anti-TARC/CCL17 antibody (Ab) can be attached directly or indirectly, e.g., via a sheep (or other species) anti-human Ab, to a solid support. Any TARC/CCL17, which is present in a sample and brought into contact with the solid support, is bound by the polyclonal, monoclonal, chimeric humanized or human anti-TARC/CCL17 Ab. A biotin-labeled mouse anti-TARC/CCL17 Ab also binds to the TARC/CCL17. Streptavidin, which is linked to horseradish peroxidase (HRPO), binds to the biotin on the mouse anti-TARC/CCL17 Ab. Upon being contacted with o-phenylenediamine, the HRPO converts the o-phenylenediamine to 2,3-diaminophenazine, which is orange-brown in color and can be measured spectrophotometrically at 492 nm. In some embodiments, the methods can use TARC/CCL17 antibodies and detection methods as described in Morita A., et al., “Evaluation of human thymus and activation-regulated chemokine concentrations in blood using a new sandwich ELISA based on monoclonal antibodies.” Clin Chim Acta 2002; 322: 67-75; or Furusyo, N., et al., “Thymus and activation regulated chemokines in children with atopic dermatitis: Kyushu University Ishigaki Atopic Dermatits Study (KIDS)” European Journal of Dermatology. (September-October 2007) 17(5):397-404, each incorporated herein by reference).

The method can further comprise diagnosing, determining a prognosis, or assessing the efficacy of a treatment (therapeutic or prophylactic) of a patient from whom the test sample was obtained. If the method further comprises assessing the efficacy of a therapeutic/prophylactic treatment of the patient from whom the test sample was obtained, the method optionally further comprises modifying the therapeutic/prophylactic treatment of the patient as needed to improve efficacy. The method can be adapted for use in an automated system or a semi-automated system.

Generally, a predetermined level can be employed as a benchmark against which to assess results obtained upon assaying a test sample for TARC/CCL17 (e.g., TARC/CCL17, fragments of TARC/CCL17, variants of TARC/CCL17, or any combinations thereof). Generally, in making such a comparison, the predetermined level is obtained by running a particular assay a sufficient number of times and under appropriate conditions such that a linkage or association of analyte presence, amount or concentration with a particular stage or endpoint of a disease, disorder or condition (e.g. RA) or with particular indicia can be made. Typically, the predetermined level is obtained with assays of reference subjects (or populations of subjects). The TARC/CCL17 measured can include fragments thereof, degradation products thereof, and/or enzymatic cleavage products thereof.

In particular, with respect to a predetermined level as employed for monitoring disease progression and/or treatment, the amount or concentration of TARC/CCL17 (e.g., TARC/CCL17, fragments of TARC/CCL17, variants of TARC/CCL17, or any combinations thereof) may be “unchanged,” “favorable” (or “favorably altered”), or “unfavorable” (or “unfavorably altered”). “Elevated” or “increased” refers to an amount or a concentration in a test sample that is higher than a typical or normal level or range (e.g., predetermined level), or is higher than another reference level or range (e.g., earlier or baseline sample). The term “lowered” or “reduced” refers to an amount or a concentration in a test sample that is lower than a typical or normal level or range (e.g., predetermined level), or is lower than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal level or range for TARC/CCL17 is defined in accordance with standard practice. A so-called altered level or alteration can be considered to have occurred when there is any net change as compared to the typical or normal level or range, or reference level or range, which cannot be explained by experimental error or sample variation. Thus, the level measured in a particular sample will be compared with the level or range of levels determined in similar samples from a so-called normal subject. In this context, a “normal subject” is an individual with no detectable disease or disorder, and a “normal” (sometimes termed “control”) patient or population is/are one(s) that exhibit(s) no detectable disease or disorder, respectively, for example. An “apparently normal subject” is one in which TARC/CCL17 has not been or is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. Thus, inter alia, the disclosure provides a method of screening for a subject having, or at risk of having, rheumatoid arthritis.

The method of assay can also involve the assay of other markers and the like as discussed herein and known in the art. For example, the method of assay can also involve the assay (detecting) of TARC/CCL17, and/or RF, anti-MCV, ACPAs, CRP, MMP3, for example.

The methods described herein also can be used to determine whether or not a subject has or is at risk of developing RA, such as discussed herein and known in the art. Specifically, such a method can comprise the steps of:

(a) determining the concentration or amount in a test sample from a subject of TARC/CCL17 (e.g., TARC/CCL17, fragments of TARC/CCL17, variants of TARC/CCL17, or any combinations thereof) using the methods described herein, or methods known in the art; and

(b) comparing the concentration or amount of TARC/CCL17 (e.g., TARC/CCL17, fragments of TARC/CCL17, variants of TARC/CCL17, or any combinations thereof) determined in step (a) with a predetermined level, wherein, if the concentration or amount of TARC/CCL17 determined in step (a) is favorable with respect to a predetermined level, then the subject is determined not to have, or to be at risk for RA as discussed herein and known in the art. However, if the concentration or amount of TARC/CCL17 determined in step (a) is unfavorable (such as for example, increased) with respect to the predetermined level, then the subject is determined to have or to be at risk for RA as discussed herein and known in the art.

Additionally, provided herein is method of monitoring the progression of disease in a subject. In some embodiments, the method comprises the steps of:

(a) determining the concentration or amount in a test sample from a subject of TARC/CCL17 (e.g., TARC/CCL17, fragments of TARC/CCL17, variants of TARC/CCL17, or any combinations thereof);

(b) determining the concentration or amount in a later test sample from the subject of TARC/CCL17; and (c) comparing the concentration or amount of TARC/CCL17 as determined in step (b) with the concentration or amount of TARC/CCL17 determined in step (a), wherein if the concentration or amount determined in step (b) is unchanged or is unfavorable (such as, for example, increased) when compared to the concentration or amount of TARC/CCL17 determined in step (a), then the disease in the subject is determined to have continued, progressed or worsened. By comparison, if the concentration or amount of TARC/CCL17 as determined in step (b) is favorable when compared to the concentration or amount of TARC/CCL17 as determined in step (a), then the disease in the subject is determined to have discontinued, regressed or improved.

Optionally, the method further comprises comparing the concentration or amount of TARC/CCL17 as determined in step (b), for example, with a predetermined level. Further, optionally the method comprises treating the subject with one or more pharmaceutical compositions for a period of time if the comparison shows that the concentration or amount of TARC/CCL17 as determined in step (b), for example, is unfavorably altered (such as, for example, increased) with respect to the predetermined level.

Still further, the methods can be used to monitor treatment in a subject receiving treatment with one or more pharmaceutical compositions. Specifically, such methods involve providing a first test sample from a subject before the subject has been administered one or more pharmaceutical compositions, such as one or more DMARDs. Next, the concentration or amount of TARC/CCL17 is determined (e.g., using the methods described herein or as known in the art) in a first test sample from a subject. After the concentration or amount of TARC/CCL17 is determined, optionally the concentration or amount of TARC/CCL17 is then compared with a predetermined level. If the concentration or amount of TARC/CCL17 as determined in the first test sample is lower than the predetermined level, then the subject is not treated with one or more pharmaceutical compositions. However, if the concentration or amount of TARC/CCL17 as determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about one (1) day to about thirty (30) days, at which time the success of the treatment can be assessed (e.g., using clinical indicators or determining the concentration or amount of TARC/CCL17 after treatment has begun).

During the course of treatment with the one or more pharmaceutical compositions, second and subsequent test samples are then obtained from the subject. The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of TARC/CCL17 (e.g., TARC/CCL17, fragments of TARC/CCL17, variants of TARC/CCL17, or any combinations thereof) is determined in the second or subsequent test sample is determined (e.g., using the methods described herein or as known in the art). The concentration or amount of TARC/CCL17 as determined in each of the second and subsequent test samples is then compared with the concentration or amount of TARC/CCL17 as determined in the first test sample (e.g., the test sample that was originally optionally compared to the predetermined level). If the concentration or amount of TARC/CCL17 as determined in step (c) is favorable when compared to the concentration or amount of TARC/CCL17 as determined in step (a), then the disease or infection in the subject is determined to have discontinued, regressed or improved, and the subject should continue to be administered the one or pharmaceutical compositions of step (b). However, if the concentration or amount determined in step (c) is unchanged or is unfavorable (such as, for example, increased) when compared to the concentration or amount of TARC/CCL17 as determined in step (a), then the disease is determined to have continued, progressed or worsened, and the subject should be treated with a higher concentration of the one or more pharmaceutical compositions administered to the subject in step (b) or the subject should be treated with one or more pharmaceutical compositions that are different from the one or more pharmaceutical compositions administered to the subject in step (b). Specifically, the subject can be treated with one or more pharmaceutical compositions that are different from the one or more pharmaceutical compositions that the subject had previously received, and evaluate the efficacy of the different composition(s) to decrease or lower said subject's TARC/CCL17 level and/or improve symptoms of the disease.

Generally, for assays in which repeat testing may be done (e.g., monitoring disease progression and/or response to treatment), a second or subsequent test sample is obtained at a period in time after the first test sample has been obtained from the subject. Specifically, a second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years, or about 10.0 years or more after the first test sample from the subject is obtained, or at least about after one of the aforementioned time periods. When used to monitor disease progression, the above assay can be used to monitor the progression of disease in subjects suffering from any conditions associated with RA. Such conditions are typically chronic, as no cure for RA exists, but such conditions can be acute, also known as critical care conditions. Acute conditions are life-threatening diseases or other critical medical conditions involving, for example, the cardiovascular, neurological, or excretory system. Typically, critical care conditions refer to those conditions requiring acute medical intervention in a hospital-based setting (including, but not limited to, the emergency room, intensive care unit, trauma center, or other emergent care setting) or administration by a paramedic or other field-based medical personnel. For critical care conditions, repeat monitoring is generally done within a shorter time frame, namely, minutes, hours or days (e.g., repeated every about 1 minute, about 5 minutes, about 10 minutes, about 15 minutes, about 30 minutes, about 45 minutes, about 60 minutes, about 2 hours, about 3 hours, about 4 hours, 4 about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days, or at least about every one of the aforementioned timeframes, and the initial assay likewise is generally done within a shorter timeframe, e.g., about minutes, hours or days of the onset of the disease or condition.

Suitably, the assays also can be used to monitor the progression of disease in subjects suffering from chronic or non-acute conditions. Non-critical care or, non-acute conditions, refers to conditions other than acute, life-threatening disease or other critical medical conditions. Typically, non-acute conditions include those of longer-term or chronic duration. For non-acute conditions, repeat monitoring generally is done with a longer timeframe, e.g., hours, days, weeks, months or years (e.g., after about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years, or more (e.g., for the lifespan of the subject)), and the initial assay likewise generally is done within a longer time frame, e.g., about hours, days, months or years of the onset of the disease or condition.

Furthermore, the above assays can be performed using a first test sample obtained from a subject where the first test sample is obtained from one source, such as urine, serum or plasma. Optionally the above assays can then be repeated using a second test sample obtained from the subject where the second test sample is obtained from another source. For example, if the first test sample was obtained from urine, the second test sample can be obtained from serum or plasma. The results obtained from the assays using the first test sample and the second test sample can be compared. The comparison can be used to assess the status of a disease or condition in the subject.

Moreover, the present disclosure also relates to methods of determining whether a subject predisposed to or suffering from a RA will benefit from treatment. In particular, the disclosure relates to TARC/CCL17 companion diagnostic methods and products. Thus, the method of “monitoring the treatment of disease in a subject” as described herein further optimally also can encompass selecting or identifying candidates for therapy, such as therapy with DMARDs, analgesics, and/or physical therapy.

Thus, in some embodiments, the disclosure also provides a method of determining whether a subject having RA, or at risk for having RA (as discussed herein and known in the art) is a candidate for an RA therapy. Generally, the subject is one who has experienced some symptom of the disease or who has actually been diagnosed as having, or being at risk for, RA, and/or who demonstrates an unfavorable concentration or amount (such as, for example, an increased concentration of TARC/CC17 when compared to a predetermined level) of TARC/CCL17 or a fragment thereof, as described herein.

The method optionally comprises an assay as described herein, where analyte is assessed before and following treatment of a subject with one or more pharmaceutical compositions, or where analyte is assessed following such treatment and the concentration or the amount of analyte is compared against a predetermined level. An unfavorable concentration (such as, for example, an increased concentration when compared to a predetermined level) of amount of analyte observed following treatment confirms that the subject will not benefit from receiving further or continued treatment, whereas a favorable concentration or amount of analyte observed following treatment confirms that the subject will benefit from receiving further or continued treatment. This confirmation assists with management of clinical studies, and provision of improved patient care.

While certain embodiments herein are advantageous when employed to assess RA, or risk of RA onset, the assays and kits also optionally can be employed to assess TARC/CCL17 in other diseases, disorders and conditions as appropriate.

Generally, any method that can detect or quantify biomarkers in a sample can be used in the methods described herein. These methods include physical and molecular biology methods in addition to immunological methods. For example, suitable physical methods include mass spectrometric methods, fluorescence resonance energy transfer (FRET) assays, chromatographic assays, and dye-detection assays. Suitable molecular biology methods that can be used include, but are not limited to, Northern or Southern blot hybridization, nucleic acid dot- or slot-blot hybridization, in situ hybridization, nucleic acid chip assays, PCR, reverse transcriptase PCR(RT-PCR), or real time PCR (taq-man PCR). Other methods to detect biomarkers include, e.g., nuclear magnetic resonance (NMR), fluorometry, colorimetry, radiometry, luminometry, or other spectrometric methods, plasmon-resonance (e.g. BIACORE), and one- or two-dimensional gel electrophoresis.

Once measured, the concentration of TARC/CCL17 and that of any other additional biomarker being assessed is compared to a predetermined reference value for the specific biomarker. A measured, i.e. determined, TARC/CCL17 concentration that exceeds the reference TARC/CCL17 value is indicative of RA or increased risk of RA in the subject. The reference value may be determined in one of several ways. For example, the TARC/CCL17 reference value can be the TARC/CCL17 concentration measured in a sample taken from a control subject, or may be the median TARC/CCL17 concentration calculated from the concentrations measured in multiple control samples taken from a group of control subjects. A median TARC/CCL17 concentration is preferably obtained from a group of at least 20 control subjects, at least 30 control subjects, or at least 40 control subjects. The predetermined reference value for the biomarker can be a predetermined cutoff value.

A “control subject” is a healthy subject, i.e. a subject having no clinical signs or symptoms of RA. Preferably a control subject is clinically evaluated for otherwise undetected signs or symptoms of RA, which evaluation may include routine physical examination and/or laboratory testing.

Alternatively, a TARC/CCL17 cutoff value (or predetermined cutoff value) can be determined by a receiver operating curve (ROC) analysis from biological samples of a patient group. ROC analysis as generally known in the biological arts is a determination of the ability of a test to discriminate one condition from another, e.g. diseased cases from normal cases, or to compare the diagnostic performance of two or more laboratory or diagnostic tests. A description of ROC analysis as applied according to the present disclosure is provided in P. J. Heagerty et al., Time-dependent ROC curves for censored survival data and a diagnostic marker, Biometrics 56:337-44 (2000), the disclosure of which is hereby incorporated by reference in its entirety. Alternatively, a TARC/CCL17 cutoff value can be determined by a quartile analysis of biological samples of a patient group. For example, a TARC/CCL17 cutoff value can be determined by selecting a value that corresponds to any value in the 25th-75th percentile range, preferably a value that corresponds to the 25th percentile, the 50th percentile or the 75th percentile, and more preferably the 75th percentile. An exemplary TARC/CCL17 reference value obtained from the median of a relevant patient group is about 24 pg/ml in serum. An exemplary TARC/CCL17 reference value obtained from quartile analysis at the 75th percentile is about 46 pg/ml in serum. Such statistical analyses can be performed using any method known in the art and can be implemented through any number of commercially available software packages (e.g., from Analyse-it Software Ltd., Leeds, UK; StataCorp LP, College Station, Tex.; SAS Institute Inc., Cary, N.C.).

In some embodiments, the methods comprise a TARC/CCL17 cutoff value of about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, or about 700 pg/mL.

The method may further include assessing at least one additional biomarker of RA, for example by measuring the concentration at least one additional biomarker in the biological sample, and comparing the measured concentration to a reference value for each additional biomarker being assessed. One, two, three, four or more additional biomarkers may be assessed. Additional such biomarkers of RA include, but are not limited to, Rheumatoid Factor (RF), C-reactive protein (CRP), matrix metalloproteinase-3 (MMP3), and anti-cyclic citrullinated peptide antibody (Anti-CCP, or anti-citrullinated peptide antibody (ACPA)). A reference value (or predetermined level) may be similarly determined for any other biomarker of RA, as described herein with respect to determining a reference value for TARC/CCL17. Typically, a measured i.e., determined concentration of any additional biomarker in a biological sample that exceeds the reference value for that biomarker is also indicative of RA or increased risk of RA onset in the subject. Instances of biomarkers for which the opposite is true are nevertheless possible, i.e. biomarkers for which the relationship between concentration in a biological sample and instance of RA or increased risk of RA onset is inverse, such that a determined biomarker concentration that is below the reference value for the biomarker is indicative of RA or increased risk of RA onset in the subject.

For example, elevated levels Rheumatoid Factor (RF) in the blood have been used as diagnostic biomarkers of RA. RF is an autoantibody against the Fc portion of IgG which together can form a RF-IgG complex that is thought to contribute to the RA disease process. RF is often evaluated in patients suspected of having any form of arthritis even though positive results can be due to other causes, and negative results do not rule out disease. In fact, about 15% of RA patients are tested as seronegative for RF. Nevertheless, in combination with signs and symptoms, RF can play a role in both diagnosis and disease prognosis, and is part of the usual disease criteria of rheumatoid arthritis. Increased levels of rheumatoid factor (generally above 20 IU/mL, 1:40, or over the 95th percentile) occur in rheumatoid arthritis (present in 80%). Typically, higher levels of RF in a sample correlate to a greater probability of destructive articular disease. Thus, embodiments of the methods described herein comprise determining the concentration of RF and TARC/CCL17 in a sample.

Similarly, the concentrations of any one or more of the other RA markers (e.g., C-reactive protein (CRP), matrix metalloproteinase-3 (MMP3), and anti-cyclic citrullinated peptide antibody (Anti-CCP)), optionally with RF, may be determined in addition to TARC/CCL17 and each compared to a corresponding predetermined reference value as described herein. For example, anti-citrullinated protein antibodies (ACPAs) or anti-CCP, are positive only in about 67% of all RA cases, but are rarely false-positive for RA. This provides a diagnostic specificity of about 95%. As with TARC/CCL17 and RF, ACPAs may be detectable even before onset of clinical disease. The most common tests for ACPAs are the anti-CCP (cyclic citrullinated peptide) test and the Anti-MCV assay (antibodies against mutated citrullinated Vimentin). Recently a serological point-of-care test (POCT) for the early detection of RA has been developed that combines detection of rheumatoid factor and anti-MCV for diagnosis of rheumatoid arthritis. However, the sensitivity and specificity of this test may be increased by its use in combination with detecting TARC/CCL17 levels in samples.

Thus, in some embodiments the methods encompass detection of TARC/CCL17 and at least one marker selected from Rheumatoid Factor (RF), C-reactive protein (CRP), matrix metalloproteinase-3 (MMP3), and anti-cyclic citrullinated peptide antibody (Anti-CCP, or anti-citrullinated peptide antibody (ACPA)). In some embodiments, the methods encompass detection of TARC/CCL17 and anti-CCP, and optionally, at least one marker selected from RF, CRP, and MMP3. In some embodiments, the methods encompass detection of TARC/CCL17 and RF and, optionally, at least one marker selected from anti-CCP, C-reactive protein (CRP), and matrix metalloproteinase-3 (MMP3). In some embodiments, the methods encompass detection of TARC/CCL17, anti-CCP, and RF and, optionally, at least one marker selected from CRP and MMP3. In some embodiments, the methods encompass detection of TARC/CCL17, anti-CCP, RF, CRP, and MMP3.

### C. KITS

Provided herein is a kit, which may be used for treating a subject suffering from RA, or at increased risk of RA, or diagnosing a subject as having RA as described previously herein.

Kits to be used for treating a patient will contain an antibody specific for TARC/CCL17. The kits preferably include instructions for treating a subject using the antibodies described herein. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Also provided is a kit for assaying a test sample for TARC/CCL17 (e.g., TARC/CCL17, fragments of TARC/CCL17, variants of TARC/CCL17, or any combinations thereof). The kit comprises at least one component for assaying the test sample for TARC/CCL17 and instructions for assaying the test sample for (e.g., TARC/CCL17, fragments of TARC/CCL17, variants of TARC/CCL17, or any combinations thereof). The at least one component includes at least one composition comprising an isolated antibody that specifically binds to (e.g., TARC/CCL17, fragments of TARC/CCL17, variants of TARC/CCL17, or any combinations thereof). The antibody has a variably heavy domain region and a variable light domain region. The antibody is optionally detectably labeled.

For example, the kit can comprise instructions for assaying the test sample for (e.g., TARC/CCL17, fragments of TARC/CCL17, variants of TARC/CCL17, or any combinations thereof) by immunoassay, e.g., chemiluminescent microparticle immunoassay. The instructions can be in paper form or computer-readable form, such as a disk, CD, DVD, or the like. The antibody can be an TARC/CCL17 capture antibody and/or a TARC/CCL17 detection antibody. Alternatively or additionally, the kit can comprise a calibrator or control, e.g., purified, and optionally lyophilized, (e.g., TARC/CCL17, fragments of TARC/CCL17, variants of TARC/CCL17, or any combinations thereof), and/or at least one container (e.g., tube, microtiter plates or strips, which can be already coated with an anti-TARC/CCL17 monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying TARC/CCL17.

Any antibodies, which are provided in the kit, such as recombinant antibodies specific for TARC/CCL17, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution.

If desired, the kit can contain a solid phase, such as a magnetic particle, bead, test tube, microtiter plate, cuvette, membrane, scaffolding molecule, film, filter paper, a quartz crystal, disc or chip. The kit may also include a detectable label that can be or is conjugated to an antibody, such as an antibody functioning as a detection antibody. The detectable label can for example be a direct label, which may be an enzyme, oligonucleotide, nanoparticle chemiluminophore, fluorophore, fluorescence quencher, chemiluminescence quencher, or biotin. Kits may optionally include any additional reagents needed for detecting the label.

If desired, the kit can further comprise one or more components, alone or in further combination with instructions, for assaying the test sample for another analyte, which can be a biomarker, such as a biomarker of RA. Examples of analytes include, but are not limited to TARC/CCL17, RF, CRP, MMP3, anti-MCV, and anti-CCP as well as other analytes and biomarkers discussed herein, or otherwise known in the art. In some embodiments one or more components for assaying a test sample for TARC/CCL17 enable the determination of the presence, amount or concentration of TARC/CCL17. A sample, such as a serum sample, can be assayed for TARC/CCL17 using TOF-MS and an internal standard.

The kit (or components thereof), as well as the method of determining the concentration of TARC/CCL17 in a test sample by an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing TARC/CCL17 is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, TARC/CCL17, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of TARC/CCL17 in the sample by means of an embedded algorithm and factory-determined calibration curve.

It will be understood that the methods and kits as described herein necessarily encompass other reagents and methods for carrying out the immunoassay. For instance, the disclosure encompasses various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an I-STAT® cartridge format, using a nucleic acid sequence linked to the signal antibody as a signal amplifier.

If desired, multiple concentrations of each antibody can be included in the kit to facilitate the generation of a standard curve to which the signal detected in the test sample can be compared. Alternatively, a standard curve can be generated by preparing dilutions of a single antibody solution provided in the kit.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples which are merely intended only to illustrate some aspects and embodiments of the disclosure, and should not be viewed as limiting to the scope of the disclosure. The disclosures of all journal references, U.S. patents and publications referred to herein are hereby incorporated by reference in their entireties.

### EXAMPLES

### Example 1

### Measurement of Biomarkers

Thirty five serum samples were purchased from CRCCC (Hyannis, Mass., USA) taken from patients having rheumatoid arthritis (RA) and not receiving treatment (n=35), and thirty five serum samples were purchased from ProMedDx (Norton, Mass., USA) taken from patients having RA and receiving treatment (n=35). Serum samples taken from healthy individuals (n=60) were purchased from ProMedDx. For each specimen, TARC/CCL17 concentration was determined using a commercially available ELISA (Enzyme-Linked Immuno Sorbent Assay) kit (Shionogi, Osaka, Japan). Concentrations of RF, CRP, MMP3, and anti-CCP were also determined in each specimen using commercially available assays (Shionogi, Osaka, Japan). All the kits were used according to the manufacturer's instructions. Statistical analyses were performed using Analyse-it version 2.20 (Analyse-it Software, Ltd., Leeds, UK).

Results

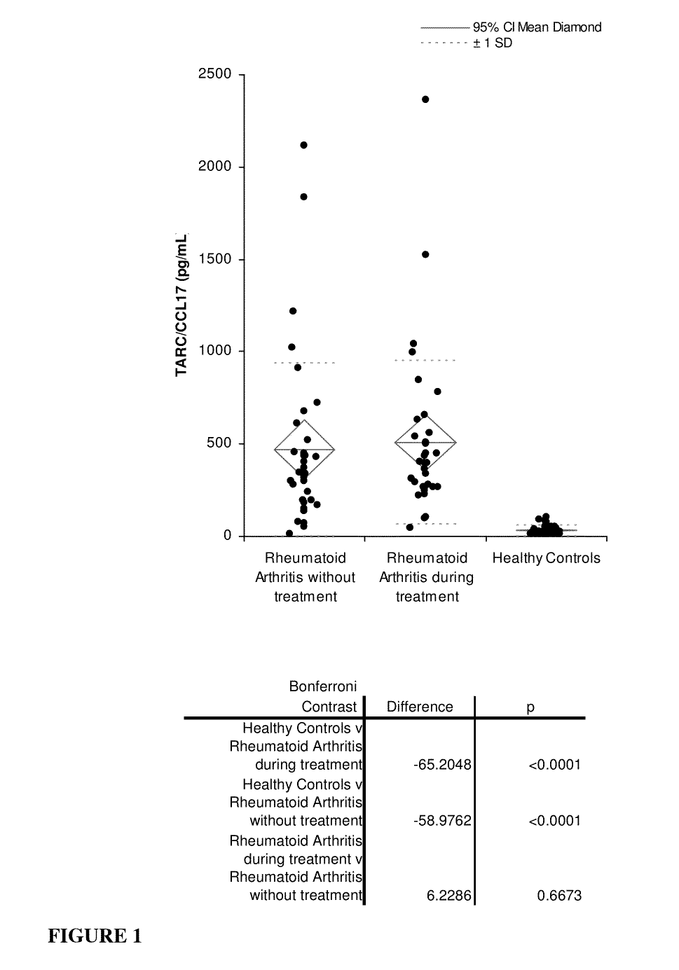

FIG. 1 depicts dot plots of serum levels of TARC/CCL17 in serum samples form patients with rheumatoid arthritis (without and during treatment) and in serum samples from healthy control patients. Table 1 summarizes the data in FIG. 1. A quartile analysis is provided in Table 2. A Kruskal-Wallis Test was used to test for differences between groups and is summarized in Table 3. The concentration of TARC/CCL17 were significantly higher in serum samples from RA patients without treatment (mean±SD: 469.5±464.8 pg/mL) and in those from RA patients during treatment (mean±SD: 510.0±438.4 pg/mL) when compared to concentration of TARC/CCL17 in healthy controls (32.1±23.8 pg/mL) (p<0.0001). There was little or no statistically significant difference between the TARC/CCL17 concentrations in samples taken from RA patients during treatment and RA patients without treatment. The solid and dotted lines represent the mean and ±1 SD, respectively.

A 95% CI (Confidence Interval) Mean Diamond was calculated for each group based on the measured TARC/CCL17 values from the samples. Dot plots and Bonferroni contrast are provided in FIG. 1. The 95% confidence interval mean shows that true mean value about the population exists within the confidence interval at the frequency of 95%. The solid horizontal line shows the mean measured TARC/CCL17 value for each sample group. As shown in FIG. 1, the mean TARC/CCL17 level in both sets of RA patients was substantially higher relative to the healthy control patients.

TABLE 1
TARC/CCL17 serum level summary.

| Sample Source | # Samples | Mean | 95% CI | SE | SD |
|---|---|---|---|---|---|
| RA without treatment | 35 | 469.5 | 309.8 to 629.2 | 78.6 | 464.8 |
| RA during treatment | 35 | 510.0 | 359.4 to 660.6 | 74.1 | 438.4 |
| Healthy Controls | 60 | 32.1 | 26.0 to 38.2 | 3.1 | 23.8 |

TABLE 2
Quartile analysis.

| Sample | # | | 1^{st} | | | 3^{rd} | | |
|---|---|---|---|---|---|---|---|---|
| Source | Samples | Min | Quartile | Median | 95% CI | Quartile | Max | IQR |
| RA | 35 | 10.00 | 182.877 | 336.524 | 239.191-438.798 | 511.236 | 2113.42 | 328.359 |
| without | | | | | | | | |
| treatment | | | | | | | | |
| RA | 35 | 44.17 | 266.860 | 395.329 | 280.257-504.815 | 554.620 | 2362.66 | 387.760 |
| during | | | | | | | | |
| treatment | | | | | | | | |
| Healthy | 60 | 10.00 | 10.00 | 24.113 | 14.505-40.688 | 45.917 | 101.50 | 35.917 |
| Controls | | | | | | | | |

TABLE 3
Kruskal-Wallis Test.

| Sample Source | # Samples | Rank Sum | Mean Rank |
|---|---|---|---|
| Healthy Controls | 60 | 1924.0 | 32.07 |
| RA during treatment | 35 | 3404.5 | 97.27 |
| RA without treatment | 35 | 3186.5 | 91.04 |
| Kruskal-Wallis' | 88.48 | | |
| statistic (H) | | | |
| X² statistic | 88.48 | | |
| DF | 2 | | |

| | |
|---|---|
| p | <0.0001 (Chi-sqr approximation, |
| | corrected for ties) |

### TARC/CCL17—Sensitive and Specific Serum Marker for RA

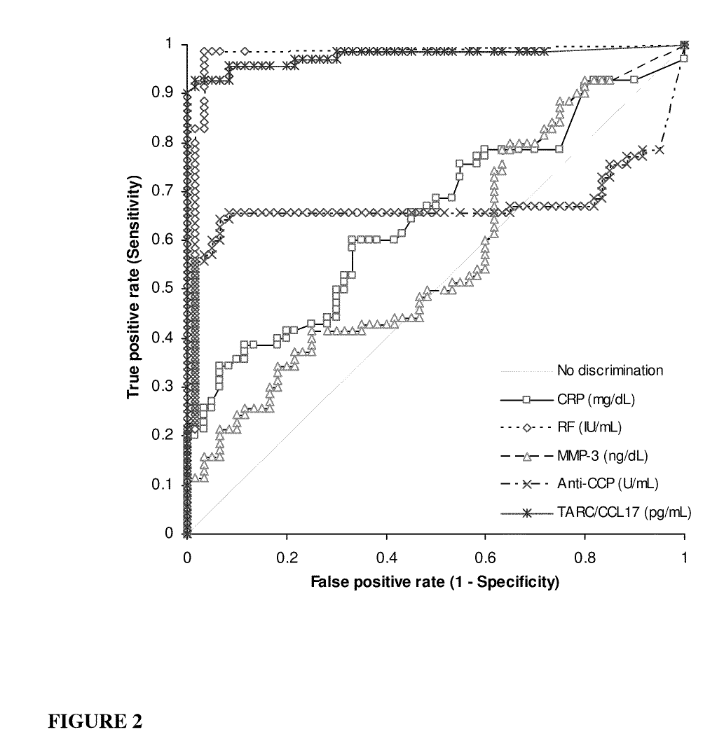

A Receiver Operating Characteristic (ROC) plot was generated from the observed true positive rate of the five measured biomarker levels in RA subjects against the observed false positive rate of the five biomarkers levels in normal subjects (FIG. 2). The diagonal line across the plot indicates the plot expected from the worst possible prediction method in which marker levels would not discriminate at all between RA subjects and normal subjects. A best possible prediction method is expected to yield a point in the upper left corner or coordinate (0,1) of the ROC space, representing 100% sensitivity (no false negatives) and 100% specificity (no false positives). Thus, an area under the curve (AUC) derived from a plot of actual data which approaches the value of 1.0 represents a best possible prediction method. As shown in FIG. 2, the ROC plot curves demonstrate the sensitivity and specificity of the TARC/CCL17 marker for RA when compared to other biomarkers associated with RA diagnosis. The combination of RA patient samples (i.e., RA patents undergoing treatment and RA patients not undergoing treatment) vs. healthy controls, provided AUC values for the biomarkers CRP (0.650), RF (0.977), MMP3 (0.575), Anti-CCP (0.673), and TARC/CCL17 (0.978). From this data TARC/CCL17 demonstrates superior sensitivity and specificity as an indicator of RA, relative to the other five RA biomarkers.

As can be seen in FIG. 2 and Table 4, the AUC of TARC/CCL17 is 0.978, indicating that elevated TARC/CCL17 level (i.e., a value of TARC/CCL17 protein above the average reference TARC/CCL17 value (−32 pg/mL) or the TARC/CCL17 cutoff value of about 100 pg/mL) is a very strong predictive classifier for RA, even when compared to rheumatoid factor (RF), which, with an AUC of 0.977 is the better of the other four biomarkers analyzed in this study. Table 5 provides a statistical analysis between the ROC curves of the various biomarkers.

TABLE 4
ROC Summary.

| Test | Area | 95% CI | SE |
|---|---|---|---|
| CRP (mg/dL) | 0.650 | 0.56 to 0.74 | 0.048 |
| RF (IU/mL) | 0.977 | 0.95 to 1.00 | 0.015 |
| MMP-3 (ng/dL) | 0.575 | 0.48 to 0.67 | 0.051 |
| Anti-CCP (U/mL) | 0.673 | 0.57 to 0.78 | 0.053 |
| TARC/CCL17 (pg/mL) | 0.978 | 0.95 to 1.00 | 0.014 |

TABLE 5
Comparison of ROC curves.

| Curves | Difference | 95% CI | SE | Z | p |
|---|---|---|---|---|---|
| CRP (mg/dL) v RF (IU/mL) | −0.33 | −0.42 to −0.23 | 0.050 | −6.56 | <0.0001 |
| CRP (mg/dL) v MMP-3 (ng/dL) | 0.08 | −0.06 to 0.21 | 0.068 | 1.11 | 0.2651 |
| CRP (mg/dL) v Anti-CCP (U/mL) | −0.02 | −0.15 to 0.11 | 0.065 | −0.35 | 0.7283 |
| CRP (mg/dL) v TARC/CCL17 (pg/mL) | −0.33 | −0.42 to −0.23 | 0.049 | −6.64 | <0.0001 |
| RF (IU/mL) v MMP-3 (ng/dL) | 0.40 | 0.30 to 0.51 | 0.053 | 7.53 | <0.0001 |
| RF (IU/mL) v Anti-CCP (U/mL) | 0.30 | 0.20 to 0.41 | 0.054 | 5.66 | <0.0001 |
| RF (IU/mL) v TARC/CCL17 (pg/mL) | 0.00 | −0.04 to 0.04 | 0.021 | −0.05 | 0.9589 |
| MMP-3 (ng/dL) v Anti-CCP (U/mL) | −0.10 | −0.25 to 0.06 | 0.079 | −1.25 | 0.2114 |
| MMP-3 (ng/dL) v TARC/CCL17 (pg/mL) | −0.40 | −0.50 to −0.30 | 0.052 | −7.81 | <0.0001 |
| Anti-CCP (U/mL) v TARC/CCL17 (pg/mL) | −0.30 | −0.42 to −0.19 | 0.057 | −5.38 | <0.0001 |

While an increase of TARC/CCL17 concentration in plasma from patients with allergic diseases, such as bronchial asthma and atopic dermatitis has been reported, this study establishes—for the first time—that elevated TARC/CCL17 concentrations are found in samples from patients with rheumatoid arthritis, relative to concentrations in samples healthy controls. The data also establishes that TARC/CCL17 shows superior diagnostic accuracy for rheumatoid arthritis, compared with other existing and clinically relevant biomarkers.

Thus, elevated levels of TARC/CCL17 can be used to diagnose rheumatoid arthritis in patient samples, or to provide a prognosis for rheumatoid arthritis risk or progression in a patient. Similarly, elevated levels of TARC/CCL17 and can be used to identify a patient as a candidate for therapy comprising one or more rheumatoid arthritis therapeutic agent(s).

### REFERENCES

Imai T, Baba M, Nishimura M, Kakizaki M, Takagi S, and Yoshie O., The T Cell-directed CC Chemokine TARC Is a Highly Specific Biological Ligand for CC Chemokine Receptor 4. JBC (1997) 272:15036-15042.
Kakinuma T, Nakamura K, Wakugawa M, Mitsui H, Tada Y, Saeki H, et al., Thymus and activation-regulated chemokine in atopic dermatitis: Serum thymus and activation-regulated chemokine level is closely related with disease activity. J Allergy Clin Immunol. (2001) 107:535-541.
Chong B F, Murphy J E, Kupper T S, and Fuhlbrigge R C., E-selectin, thymus- and activation-regulated chemokine/CCL17, and intercellular adhesion molecule-1 are constitutively coexpressed in dermal microvessels: a foundation for a cutaneous immunosurveillance system. J Immunol (2004) 172:1575-1581.
Takeuchi H, Yamamoto Y, Kitano H, and Enomoto T. Changes in thymus- and activation regulated chemokine (TARC) associated with allergen immunotherapy in patients with perennial allergic rhinitis. J Invest Allergol Clin Immunol (2005) 15:172-176.
Furusyo N, Takeoka H, Toyoda K, Murata M, Maeda S, Ohnishi H, et al. Thymus and activation regulated chemokines in children with atopic dermatitis: Kyushu University Ishigaki Atopic Dermatitis Study (KIDS). Eur J Dermatol (2007) 17:397-404.
WO 2005/005474 or US 2006/0134706 (Shionogi & Co., Ltd.) Atopic dermatitis inducer.
Package inserts of commercial reagent kits from R&D Systems (research use only), and Shionogi (IVD for a diagnosis of atopic dermatitis)

What is claimed is:

1. A method for providing a diagnosis, prognosis or risk classification of a subject having or at risk of having rheumatoid arthritis, the method comprising the steps of:
a. determining the concentration of TARC/CCL17 in a biological sample of the subject; and
b. comparing the TARC/CCL17 concentration from the sample to a reference TARC/CCL17 concentration value, wherein a TARC/CCL17 concentration in the sample greater than reference TARC/CCL17 concentration value indicates the subject has rheumatoid arthritis or has an increased risk of developing rheumatoid arthritis.

2. The method of claim 1, further comprising determining the concentration of at least one additional biomarker of rheumatoid arthritis in the sample; and comparing the concentration of the at least one additional biomarker to a reference concentration value for the at least one biomarker.

3. The method of claim 2, wherein the additional biomarker of rheumatoid arthritis is selected from Rheumatoid Factor (RF), C-reactive protein (CRP), matrix metalloproteinase-3 (MMP3), anti-cyclic citrullinated peptide antibody (Anti-CCP), and mutated citrullinated vimentin (MCV).

4. The method of any of the preceding claims, wherein the reference TARC/CCL17 concentration value is a TARC/CCL17 concentration value of a control sample, or a TARC/CCL17 cutoff value.

5. The method of claim 4, wherein the reference TARC/CCL17 concentration value is the TARC/CCL17 concentration of a control sample selected from a biological sample of a control subject and a TARC/CCL17 concentration standard.

6. The method of claim 4, wherein the reference TARC/CCL17 concentration value is selected from the TARC/CCL17 concentration value of a control sample, and the median or the mean TARC/CCL17 concentration of a plurality of control samples from a group of control subjects.

7. The method of claim 6, wherein the determined TARC/CCL17 concentration of the subject is at least double the reference TARC/CCL17 concentration value.

8. The method of claim 4, wherein the reference TARC/CCL17 concentration value is the TARC/CCL17 cutoff value determined by a receiver operating curve (ROC) analysis from biological samples of a patient group.

9. The method of claim 4, wherein the reference TARC/CCL17 concentration value is the TARC/CCL17 cutoff value determined by a quartile analysis of biological samples of a patient group.

10. The method of claim 4, wherein the reference TARC/CCL17 concentration value is the TARC/CCL17 cutoff value, and is about 32 pg/mL in serum.

11. The method of any of the preceding claims, wherein the TARC/CCL17 concentration is the TARC/CCL17 concentration in serum.

12. The method of any of the preceding claims, wherein the method further comprises providing a diagnosis of rheumatoid arthritis.

13. The method of any of the preceding claims, wherein the method comprises providing a prognosis selected from determining the severity of rheumatoid arthritis in the subject and a likelihood that the subject will develop rheumatoid arthritis.

14. The method of any of the preceding claims, wherein the subject is a human.

15. The method of claim 5, wherein the reference TARC/CCL17 concentration value is from a biological sample of a human control subject.

16. The method of any of the preceding claims, wherein the biological sample of a subject is selected from a tissue sample, bodily fluid, whole blood, plasma, serum, urine, bronchoalveolar lavage fluid, and a cell culture suspension or fraction thereof.

17. The method of any of the preceding claims, wherein the biological sample of a subject is blood plasma or blood serum.

18. The method of any of the preceding claims, wherein determining the concentration of TARC/CCL17 comprises an immunological method with molecules binding to the TARC/CCL 17.

19. The method of claim 18, wherein the molecules binding to the TARC/CCL17 comprises at least one antibody capable of specifically binding TARC/CCL17.

20. A method for the diagnosis, prognosis and/or risk stratification of rheumatoid arthritis in a subject having or suspected of having rheumatoid arthritis, the method comprising the step of detecting an increased TARC/CCL17 concentration in the subject relative to a control subject not having rheumatoid arthritis.

21. A kit for performing the method of any of the preceding claims, the kit comprising:
a. at least one reagent capable of specifically binding TARC/CCL17 to quantify the TARC/CCL17 concentration in the biological sample of a subject; and
b. a reference standard indicating a reference TARC/CCL17 concentration.

22. The kit of claim 21, further comprising at least one additional reagent capable of binding at least one additional biomarker of rheumatoid arthritis in the biological sample to quantify the concentration of the at least one additional biomarker in the biological sample, and a reference standard indicating a reference concentration of the at least one additional biomarker of rheumatoid arthritis in the biological sample.

23. The kit of claim 21, wherein the at least one reagent comprises at least one antibody capable of specifically binding TARC/CCL17.
